(19) United States
(12) Patent Application Publication
(10) Pub. No.: US 2024/0375038 A1
(43) Pub. Date: Nov. 14, 2024

(54) METHOD OF REDUCING DIMETHYL ETHER FORMATION DURING A REGENERATION CYCLE

(71) Applicant: BASF Corporation, Florham Park, NJ (US)

(72) Inventors: Nele KLINKENBERG, Nienburg/Weser (DE); Tobias ECKHARDT, Nienburg/Weser (DE); William B. DOLAN, Iselin, NJ (US); Mark TOMCZAK, Iselin, NJ (US); Justin PAN, Iselin, NJ (US)

(21) Appl. No.: 18/690,420

(22) PCT Filed: Sep. 8, 2022
(86) PCT No.: PCT/US2022/042888

§ 371 Date: Mar. 8, 2024

Related U.S. Application Data
(60) Provisional application No. 63/243,643, filed on Sep. 13, 2021.

(51) Int. Cl. B01D 53/04

(52) U.S. Cl. CPC B01D 53/0462; B01D 2253/104; B01D 2253/108; B01D 2256/22; B01D 2256/24; B01D 2257/708

(57) ABSTRACT

Disclosed in certain embodiments are methods of removing methanol from a natural gas stream comprising methanol during an adsorption step of an adsorption cycle.

### CROSS-REFERENCE TO RELATED APPLICATION(S)

This application claims the benefit of priority of U.S. Provisional Patent Application No. 63/243,643, filed on Sep. 13, 2021, the disclosure of which is hereby incorporated by reference herein in its entirety.

### BACKGROUND

Dehydration of natural gas to cryogenic specifications is critical in the pretreatment process for liquified natural gas (LNG) production. Zeolitic molecular sieves are used in such processes because they allow for the natural gas to meet the required dewpoint for liquefaction. Failure to reach this required dewpoint may result in the inability to maintain the necessary gas flow to the liquefaction section, which can constrain or shutdown the production of LNG.

Hydrothermal damage and retrograde condensation in dehydrator vessels during regeneration and adsorption lead to degradation of the molecular sieve adsorbent through leaching of the clay binder and loss of adsorption capacity. In addition, the presence of excess methanol during the regeneration cycle of an adsorption process can lead to the formation of dimethyl ether, which may also have a deleterious effect on the molecular sieve. Such effects can result in an increase in pressure drop and an uneven distribution of adsorption and/or regeneration flow, ultimately requiring premature replacement of the adsorbent.

### BRIEF DESCRIPTION OF THE DRAWINGS

The present disclosure is illustrated by way of example, and not by way of limitation, in the figures of the accompanying drawings, in which:

FIG. 1 illustrates an adsorber unit in accordance with at least one embodiment of the disclosure:

FIG. 2A illustrates an adsorber unit that includes an adsorbent bed with two adsorbent layers in accordance with at least one embodiment of the disclosure:

FIG. 2B illustrates a variation of the configuration of FIG. 2A which includes multiple adsorber units in accordance with at least one embodiment of the disclosure:

FIG. 3A illustrates a further adsorber unit in accordance with at least one embodiment of the disclosure:

FIG. 3B illustrates a variation of the configuration of FIG. 3A which includes multiple adsorber units in accordance with at least one embodiment of the disclosure:

FIG. 4 illustrates a method of treating a natural gas stream to remove methanol and reduce or eliminate formation of dimethyl ether in accordance with an embodiment of the disclosure; and

FIG. 5 is a plot showing methanol breakthrough for two different adsorbent beds.

### SUMMARY

The following presents a simplified summary of various aspects of the present disclosure in order to provide a basic understanding of such aspects. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In one aspect, a method of treating a gas stream to remove methanol and reduce or eliminate formation of dimethyl ether during a regeneration cycle comprises: directing, during an adsorption cycle of an adsorption process, the gas stream having an initial methanol mole fraction toward a first adsorbent bed of a first adsorber unit, the first adsorbent bed comprising a first adsorbent layer comprising a silica adsorbent. In at least one embodiment, an alumina content of the first adsorbent layer is about 3.1 wt. % or less based on a total weight of the first adsorbent layer, and/or the initial methanol mole fraction is from about 50 ppm to about 1000 ppm, from about 100 ppm to about 1000 ppm, from about 150 ppm to about 1000 ppm, from about 250 ppm to about 1000 ppm, from about 350 ppm to about 1000 ppm, or from about 450 ppm to about 1000 ppm.

In at least one embodiment, the alumina content of the first adsorbent layer is about 3.0 wt. % or less, about 2.9 wt. % or less, about 2.8 wt. % or less, about 2.7 wt. % or less, about 2.6 wt. % or less, about 2.5 wt. % or less, about 2.4 wt. % or less, about 2.3 wt. % or less, about 2.2 wt. % or less, about 2.1 wt. % or less, about 2.0 wt. % or less, about 1.9 wt. % or less, about 1.8 wt. % or less, about 1.7 wt. % or less, about 1.6 wt. % or less, about 1.5 wt. % or less, about 1.4 wt. % or less, about 1.3 wt. % or less, about 1.2 wt. % or less, about 1.1 wt. % or less, about 1.0 wt. % or less, 0.9 wt. % or less, about 0.8 wt. % or less, about 0.7 wt. % or less, about 0.6 wt. % or less, about 0.5 wt. % or less, about 0.4 wt. % or less, about 0.3 wt. % or less, about 0.2 wt. % or less, about 0.1 wt. % or less.

In at least one embodiment, the first adsorbent layer is substantially free of alumina.

In at least one embodiment, the method further comprises: directing, during the regeneration cycle, at least a portion of the treated gas stream through the first adsorbent bed of the first adsorber unit. In at least one embodiment, a conversion of total methanol adsorbed in the first adsorbent bed into dimethyl ether for the regeneration cycle is less than 3%, less than 7%, less than 4%, less than 1%, or less than 0.4%. In at least one embodiment, the first adsorbent bed is thermally regenerated during the regeneration cycle.

In at least one embodiment, the first adsorbent bed further comprises a second adsorbent layer comprising a zeolite. In at least one embodiment, the second adsorbent layer is downstream from the first adsorbent layer.

In at least one embodiment, the method further comprises: directing the gas stream from the first adsorber unit toward a second adsorbent bed of a second adsorber unit, the second adsorbent bed comprising a second adsorbent layer comprising a zeolite.

In at least one embodiment, a methanol mole fraction of the gas stream is reduced to about 40 ppm or less, about 30 ppm or less, about 20 ppm or less, about 10 ppm or less, about 5 ppm or less, or about 2 ppm or less prior to the gas stream contacting the second adsorbent layer.

In at least one embodiment, a water mole fraction of the gas stream is reduced to about 80 ppm or less, about 70 ppm or less, about 60 ppm or less, about 50 ppm or less, about 40 ppm or less, about 30 ppm or less, about 20 ppm or less, about 10 ppm or less, about 5 ppm or less, or about 2 ppm or less prior to the gas stream contacting the second adsorbent layer.

In at least one embodiment, a water mole fraction of the gas stream is reduced to about 1 ppm or less prior to the gas stream leaving the second adsorber unit.

In at least one embodiment, the zeolite comprises one or more of zeolite A, zeolite X, or zeolite Y.

In at least one embodiment, the second adsorbent layer comprises one or more of zeolite 3A, zeolite 4A or zeolite 5A.

In at least one embodiment, the second adsorbent layer comprises zeolite 4A.

In at least one embodiment, the zeolite is exchanged with an element selected from Li, Na, K, Mg, Ca, Sr, or Ba.

In at least one embodiment, a final methanol mole fraction of the gas stream leaving the first adsorber unit is about 20 ppm or less, about 15 ppm or less, about 10 ppm or less, about 5 ppm or less, about 4 ppm or less, about 3 ppm or less, about 2 ppm or less, about 1 ppm or less, about 0.5 ppm or less, about 0.4 ppm or less, about 0.3 ppm or less, about 0.2 ppm or less, or below 0.1 or less.

In at least one embodiment, the gas stream is a natural gas stream. In at least one embodiment, the method further comprises: forming a liquefied natural gas product from the treated natural gas stream after leaving the first adsorber unit. In at least one embodiment, the method further comprises: forming a natural gas liquid product from the treated natural gas stream after leaving the first adsorber unit. In at least one embodiment, the method further comprises: directing the natural gas stream after leaving the first adsorber unit to a natural gas pipeline.

In at least one embodiment, the method is performed as part of a dehydration process. In at least one embodiment, a water mole fraction of the gas stream is about 80 ppm or less, about 70 ppm or less, about 60 ppm or less, about 50 ppm or less, about 40 ppm or less, about 30 ppm or less, about 20 ppm or less, about 10 ppm or less, or about 5 ppm or less.

In at least one embodiment, the gas stream comprises predominately CO₂.

In another aspect, a method of treating a gas stream to remove methanol and reduce or eliminate formation of dimethyl ether during a regeneration cycle comprises: directing, during an adsorption cycle of an adsorption process, the gas stream having an initial methanol mole fraction toward a first adsorbent bed of a first adsorber unit, the first adsorbent bed comprising a first adsorbent layer comprising a silica adsorbent. In at least one embodiment, the initial methanol mole fraction is from about 250 ppm to about 1000 ppm, and a conversion of total methanol adsorbed in the first adsorbent bed into dimethyl ether for the regeneration cycle is less than 7%.

In at least one embodiment, the first adsorbent bed is thermally regenerated during the regeneration cycle.

In at least one embodiment, the first adsorbent bed further comprises a second adsorbent layer comprising a zeolite. In at least one embodiment, the second adsorbent layer is downstream from the first adsorbent layer.

In at least one embodiment, the method further comprises: directing the gas stream from the first adsorber unit toward a second adsorbent bed of a second adsorber unit, the second adsorbent bed comprising a second adsorbent layer comprising a zeolite.

In at least one embodiment, a methanol mole fraction of the gas stream is reduced to about 40 ppm or less, about 30 ppm or less, about 20 ppm or less, about 10 ppm or less, about 5 ppm or less, or about 2 ppm or less prior to the gas stream contacting the second adsorbent layer.

In at least one embodiment, a water mole fraction of the gas stream is reduced to about 80 ppm or less, about 70 ppm or less, about 60 ppm or less, about 50 ppm or less, about 40 ppm or less, about 30 ppm or less, about 20 ppm or less, about 10 ppm or less, about 5 ppm or less, or about 2 ppm or less prior to the gas stream contacting the second adsorbent layer.

In at least one embodiment, a water mole fraction of the gas stream is reduced to about 1 ppm or less prior to the gas stream leaving the second adsorber unit.

In at least one embodiment, the zeolite comprises one or more of zeolite A, zeolite X, or zeolite Y.

In at least one embodiment, the second adsorbent layer comprises one or more of zeolite 3A, zeolite 4A or zeolite 5A.

In at least one embodiment, the second adsorbent layer comprises zeolite 4A.

In at least one embodiment, the zeolite is exchanged with an element selected from Li, Na, K, Mg, Ca, Sr, or Ba.

In at least one embodiment, a final methanol mole fraction of the gas stream leaving the first adsorber unit is about 20 ppm or less, about 15 ppm or less, about 10 ppm or less, about 5 ppm or less, about 4 ppm or less, about 3 ppm or less, about 2 ppm or less, about 1 ppm or less, about 0.5 ppm or less, about 0.4 ppm or less, about 0.3 ppm or less, about 0.2 ppm or less, or below 0.1 or less.

In at least one embodiment, the gas stream is a natural gas stream.

In at least one embodiment, the method further comprises forming a liquefied natural gas product from the treated natural gas stream after leaving the first adsorber unit.

In at least one embodiment, the method further comprises forming a natural gas liquid product from the treated natural gas stream after leaving the first adsorber unit.

In at least one embodiment, the method further comprises directing the natural gas stream after leaving the first adsorber unit to a natural gas pipeline.

In at least one embodiment, the method is performed as part of a dehydration process. In at least one embodiment, a water mole fraction of the gas stream is about 80 ppm or less, about 70 ppm or less, about 60 ppm or less, about 50 ppm or less, about 40 ppm or less, about 30 ppm or less, about 20 ppm or less, about 10 ppm or less, or about 5 ppm or less.

In at least one embodiment, the gas stream comprises predominately CO₂.

In another aspect, a thermal swing adsorption system is configured to perform any of the foregoing methods.

In another aspect, a natural gas purification system comprises the thermal swing adsorption system.

### DETAILED DESCRIPTION

The present disclosure relates generally to methods of removing methanol from a gas feed stream, such as a natural gas stream, comprising methanol during an adsorption step of an adsorption cycle, as well as to adsorbent beds adapted for the same. Some embodiments relate to a single adsorber unit for removing both hydrocarbons (e.g., aliphatic C5+ hydrocarbons and mercaptans and C6+ aromatic and aliphatic hydrocarbons and mercaptans) and methanol, as well as for removing water down to cryogenic specifications for producing liquefied natural gas (LNG), rather than utilizing two or more separate adsorber units. Other embodiments relate to the use of multiple adsorber units for performing the same.

In general, molecular sieves, such as 4A and 3A zeolites, are often used to dry natural gas streams. Although these materials beneficially remove water from natural gas at the conditions of the operating units (i.e., high pressure methane and high water concentration), they are subject to hydrothermal damage. While there are other mechanisms that can damage the sieves (e.g., refluxing) which may be mitigated, hydrothermal damage appears unavoidable. Silica-based materials have been shown to be highly robust in this application with practical field experience where the adsorbent has lasted more than ten years in comparable environments: however, these materials are generally not used to remove water to cryogenic specifications required for forming liquefied natural gas.

Some embodiments described herein advantageously utilize an amorphous silica adsorbent, an amorphous silica-alumina adsorbent, a high-silica zeolite adsorbent (e.g., beta zeolite. ZSM-5, high-silica Y zeolite, etc.), or combinations thereof, with a less hydrothermally stable adsorbent (e.g., zeolite 3A, zeolite 4A, or zeolite 5A) as separate adsorbent layers to produce a robust, longer-lasting adsorbent system. In such embodiments, the mole fractions of water entering the section of an adsorbent bed containing the less hydrothermally stable adsorbent is reduced by the upstream layer of the adsorbent bed. Since there is lower mole fraction of water entering the less hydrothermally stable adsorbent during the adsorption step, there is also less water to desorb during the regeneration step and hence a lower steaming environment is created during regeneration. This is advantageous as it is known to those skilled in the art that a steaming environment can damage zeolites. While adsorbent layers may be distributed across multiple adsorbent beds in different adsorber units, some embodiments can advantageously allow for hydrocarbon adsorption and water adsorption to be performed in a single adsorber unit while being able to reduce the water mole fraction below a cryogenic maximum. This reduces the total number of adsorber units needed, thus reducing the physical size of the natural gas processing facility.

In some embodiments, the gas feed stream may comprise methanol, as well as CO₂ and H₂S which can result in the formation of carbonyl sulfide (COS) in the zeolite layer and have a deleterious effect on its performance. Similar to the reduction of water mole fraction, one or more upstream adsorbent layers may be utilized to reduce a methanol mole fraction that is exposed to the zeolite layer(s). In some embodiments, the methanol fraction leaving the adsorber unit may be significantly reduced, for example, below 1 ppm. The embodiments described herein are particularly advantageous when a natural gas stream includes a relatively high amount of methanol (e.g., greater than 200 ppm methanol) in order to reduce or prohibit the formation of dimethyl ether (DME) in the adsorbent bed during a regeneration cycle.

The adsorption process of the present disclosure, used to remove methanol, heavy hydrocarbons (e.g., C5+ or C6+ components), and/or water from gas feed streams (e.g., a natural gas streams), may be accomplished by thermal swing adsorption (TSA). TSA processes are generally known in the art for various types of adsorptive separations. Generally. TSA processes utilize the process steps of adsorption at a low temperature, regeneration at an elevated temperature with a hot purge gas, and a subsequent cooling down to the adsorption temperature. TSA processes are often used for drying gases and liquids and for purification where trace impurities are to be removed. TSA processes are often employed when the components to be adsorbed are strongly adsorbed on the adsorbent, and thus heat is required for regeneration.

A typical TSA process includes adsorption cycles and regeneration (desorption) cycles, each of which may include multiple adsorption steps and regeneration steps, as well as cooling steps and heating steps. The regeneration temperature is higher than the adsorption temperature in order to effect desorption of water, methanol, and heavy hydrocarbons. To illustrate, during the first adsorption step, which employs an adsorbent for the adsorption of C5+ or C6+ components from a gas stream (e.g., a raw natural gas stream), the temperature is maintained at less than 150° F. (66° C.) in some embodiments, and from about 60° F. (16° C.) to about 120° F. (49° C.) in other embodiments. In the regeneration step of the present disclosure, water and the C5+ or C6+ components adsorbed in the adsorbent bed initially are released from the adsorbent bed, thus regenerating the adsorbent at temperatures from about 300° F. (149° C.) to about 550° F. (288° C.) in some embodiments.

In the regeneration step, part of one of the gas streams (e.g., a stream of natural gas), the product effluent from the adsorber unit, or a waste stream from a downstream process can be heated, and the heated stream is circulated through the adsorbent bed to desorb the adsorbed components. In some embodiments, it is advantageous to employ a hot purge stream comprising a heated raw natural gas stream for regeneration of the adsorbent.

In some embodiments, the pressures used during the adsorption and regeneration steps are generally elevated at typically 700 to 1500 psig. Typically, heavy hydrocarbon adsorption is carried out at pressures close to that of the feed stream and the regeneration steps may be conducted at about the adsorption pressure or at a reduced pressure. When a portion of an adsorption effluent stream is used as a purge gas, the regeneration may be advantageously conducted at about the adsorption pressure, especially when the waste or purge stream is re-introduced into the raw natural gas stream, for example.

As used herein, a “mercaptan” refers to an organic sulfur-containing compound including, but not limited to, methyl mercaptans (C1-RSH), ethyl mercaptans (C2-RSH), propyl mercaptans (C3-RSH), butyl mercaptans (C4-RSH), dimethyl sulfide (DMS), and dimethyl disulfide (DMDS).

While embodiments of the present disclosure are described with respect to natural gas purification processes, it is to be understood by those of ordinary skill in the art that the embodiments herein may be utilized in or adapted for use in other types of industrial applications that require methanol and/or water removal in addition to LNG and natural gas liquid (NGL) applications.

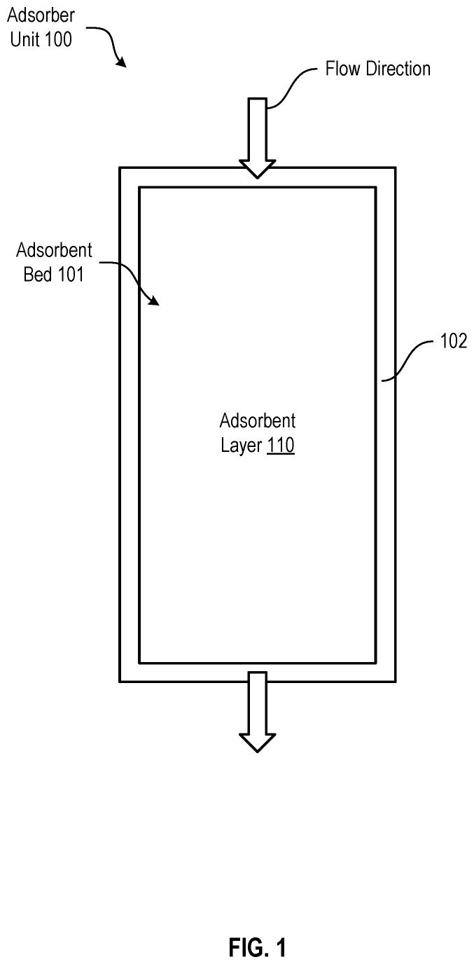

FIG. 1 illustrates an adsorber unit 100 in accordance with at least one embodiment of the disclosure. In some embodiments, the adsorber unit 100 includes a single vessel 102 that houses an adsorbent bed 101. Other embodiments may utilize multiple vessels and adsorbent beds, for example, when implementing a continuous TSA process where one or more adsorbent beds are subject to an adsorption cycle while one or more beds are subject to a regeneration cycle. For example, the adsorber unit 100 may include, in some embodiments, two or more vessels and adsorbent beds that are duplicates of the vessel 102 and the adsorbent bed 101 (not shown). While the adsorbent bed 101 is subjected to an adsorption cycle, a duplicate adsorbent bed is subjected to a regeneration cycle, for example, using a product gas resulting from the adsorption cycle performed with the adsorbent bed 101.

The adsorbent bed 101 includes adsorbent layer 110 contained inside a vessel 102. The flow direction indicates the flow of a gas feed stream through an inlet of the vessel 102 and through the adsorbent layer 110 before reaching an outlet of the vessel 102. In some embodiments, the adsorbent layer 110 may comprise its adsorbent material in a form of adsorbent beads having diameters, for example, from about 1 mm to about 5 mm.

In some embodiments, the adsorbent layer 110 comprises an adsorbent that is preferentially selective for C5+ or C6+ hydrocarbons. As used herein, the terms “preferentially selective for” or “selective for” indicates that the adsorbent adsorbs the specified compound at a greater equilibrium loading compared to methane, further described by the following equation: selectivity=(loading C6+/concentration C6+)/(loading C1/concentration C1), where C1 is methane, and where loading is defined as moles of component adsorbed/gram of adsorbent. In certain embodiments, C5+ or C6+ compounds may comprise one or more of pentane, hexane, benzene, heptane, octane, nonane, toluene, ethylbenzene, xylene, or neopentane. In some embodiments, the adsorbent layer 110 is able to at least partially adsorb methanol and water from a feed gas stream comprising the same.

In some embodiments, the adsorbent layer 110 comprises a silica adsorbent, a silica-alumina adsorbent, or a high-silica zeolite adsorbent. In some embodiments, the adsorbent layer 110 comprises an amorphous silica adsorbent and/or an amorphous silica-alumina adsorbent. Amorphous silica adsorbents and amorphous silica-alumina adsorbents may be at least partially crystalline. In some embodiments, an amorphous silica adsorbents or an amorphous silica-alumina adsorbent may be at least 50% amorphous, at least 60% amorphous, at least 70% amorphous, at least 80% amorphous, at least 90% amorphous, or 100% amorphous. In some embodiments, an amorphous silica adsorbents or an amorphous silica-alumina adsorbent may further include other components, such as adsorbed cations. An exemplary adsorbent for use in the adsorbent layer 110 may be Durasorb™ HC (available from BASF).

In some embodiments, the adsorbent layer 110 comprises a high-silica zeolite adsorbent, such as beta zeolite, ZSM-5, Y zeolite, or combinations thereof. As used herein, “high-silica zeolite” refers to a material having a silica-to-alumina ratio, on a molar basis, of at least 5, of at least 10, of at least 20, at least 30, at least 50, at least 100, at least 150, at least 200, at least 250, at least 300, at least 350, at least 400, at least 450, or at least 500, or within any range defined therebetween (e.g., 5 to 500, 10 to 500, 10 to 400, 20 to 300, etc.). In some embodiments, the silica to alumina ratio is in the range of from 20 to 500.

In some embodiments, the adsorbent layer 110 is a microporous adsorbent comprising silica and/or alumina. As used herein, the term “microporous adsorbent” refers to an adsorbent material having one or more of the following properties: a relative micropore surface area (RMA), which is the ratio of micropore surface area to Brunauer-Emmett-Teller (BET) surface area, that is greater than 5%, greater than 10%, greater than 15%, greater than 20%, greater than 25%, or greater than 30%: a total pore volume for pores between 500 nm and 20000 nm in diameter, as measured via mercury porosimetry, that is greater than 5 mm³/g, greater than 10 mm³/g, greater than 20 mm³/g, greater than 30 mm³/g, greater than 40 mm³/g, greater than 45 mm³/g, or greater than 50 mm³/g: a pore volume (e.g., Barrett-Joyner-Halenda (BJH) pore volume) that is greater than 0.40 cm³/g, is greater than 0.40 cm³/g and less than 0.50 cm³/g, or is greater than 0.425 cm³/g and less than 0.475 cm³/g; and/or a BET surface area greater than 400 m²/g, greater than 500 m²/g, greater than 600 m²/g, greater than 700 m²/g, greater than 800 m²/g, or greater than 900 m²/g, Micropore surface area and BET surface area can be characterized via nitrogen porosimetry using, for example, a Micromeritics ASAPR 2000 porosimetry system using Micromeritics ASAPR: 2010 software for analysis. Mercury porosimetry can be performed using, for example, a Thermo Scientific™ Pascal 140/240 porosimeter. Resulting porosity data can be analyzed using, for example, Pascal 140/240/440 v. 1.05 software.

As used herein, “micropore surface area” refers to total surface area associated with pores below 200 Angstroms in diameter. In some embodiments, a micropore surface area of the microporous adsorbent is greater than 40 m²/g, greater than 50 m²/g, greater than 100 m²/g, greater than 150 m²/g, greater than 200 m²/g, or greater than 230 m²/g. In some embodiments, the micropore surface area of the microporous adsorbent is from 40 m²/g to 300 m²/g, from 50 m²/g to 300 m²/g, from 100 m²/g to 300 m²/g, from 150 m²/g to 300 m²/g, from 200 m²/g to 300 m²/g, or from 230 m²/g to 300 m²/g. In some embodiments, a relative micropore surface area is from about 5% to about 10%, about 10% to about 15%, about 15% to about 20%, about 20% to about 25%, about 25% to about 30%, or in any range defined therebetween (e.g., about 15% to about 25%). In some embodiments, a corresponding BET surface area of the microporous adsorbent ranges from about 650 m²/to about 850 m²/g.

In some embodiments, the microporous adsorbent comprises amorphous SiO₂ at a weight percent greater than 85%, greater than 90%, greater than 95%, greater than 96%, greater than 97%, greater than 98%, or greater than 99%. In some embodiments, the microporous adsorbent further comprises Al₂O₃ at a weight percent of up to 20% (i.e., from greater than 0% to 20%), up to 15%, up to 10%, up to 9%, up to 8%, up to 7%, up to 6%, up to 5%, up to 4%, up to 3%, up to 2%, or up to 1%.

In some embodiments, the total pore volume for pores between 500 nm and 20000 nm in diameter of the microporous adsorbent is greater than 20 mm³/g, greater than 40 mm³/g, greater than 70 mm³/g, greater than 100 mm³/g, greater than 120 mm³/g, greater than 140 mm³/g, greater than 150 mm³/g, greater than 160 mm³/g, or greater than 170 mm³/g. In some embodiments, the total pore volume for pores between 500 nm and 20000 nm in diameter of the microporous adsorbent is from 20 mm³/g to 200 mm³/g, from 40 mm³/g to 200 mm³/g, from 70 mm³/g to 200 mm³/g, from 100 mm³/g to 200 mm³/g, from 120 mm³/g to 200 mm³/g, from 140 mm³/g to 200 mm³/g, from 150 mm³/g to 200 mm³/g, from 160 mm³/g to 200 mm³/g, or from 170 mm³/g to 200 mm³/g.

In some embodiments, the BET surface area of the microporous adsorbent is from 400 m²/g to 1000 m²/g, from 500 m²/g to 1000 m²/g, from 600 m²/g to 1000 m²/g, from 700 m²/g to 1000 m²/g, from 800 m²/g to 1000 m²/g, or from 900 m²/g to 1000 m²/g.

In some embodiments, a bulk density of the microporous adsorbent is less than 600 kg/m³. In some embodiments, a bulk density of the microporous adsorbent is at least 600 kg/m³, from about 600 kg/m³ to about 650 kg/m³, about 650 kg/m³ to about 700 kg/m³, about 700 kg/m³ to about 750 kg/m³, about 750 kg/m³ to about 800 kg/m³, about 850 kg/m³ to about 900 kg/m³, about 950 kg/m³ to about 1000 kg/m³, or in any range defined therebetween.

In some embodiments, the adsorbent layer comprises an adsorbent that has an alumina content of about 4.0 wt. % or less, where weight percent is computed based on a total weight of the adsorbent. In some embodiments, the adsorbent has an alumina content of about 3.9 wt. % or less, about 3.8 wt. % or less, about 3.7 wt. % or less, about 3.6 wt. % or less, about 3.5 wt. % or less, about 3.4 wt. % or less, about 3.3 wt. % or less, about 3.2 wt. % or less, about 3.1 wt. % or less, about 3.0 wt. % or less, about 2.9 wt. % or less, about 2.8 wt. % or less, about 2.7 wt. % or less, about 2.6 wt. % or less, about 2.5 wt. % or less, about 2.4 wt. % or less, about 2.3 wt. % or less, about 2.2 wt. % or less, about 2.1 wt. % or less, about 2.0 wt. % or less, about 1.9 wt. % or less, about 1.8 wt. % or less, about 1.7 wt. % or less, about 1.6 wt. % or less, about 1.5 wt. % or less, about 1.4 wt. % or less, about 1.3 wt. % or less, about 1.2 wt. % or less, about 1.1 wt. % or less, about 1.0 wt. % or less, 0.9 wt. % or less, about 0.8 wt. % or less, about 0.7 wt. % or less, about 0.6 wt. % or less, about 0.5 wt. % or less, about 0.4 wt. % or less, about 0.3 wt. % or less, about 0.2 wt. % or less, about 0.1 wt. % or less, or within any range defined between any of the foregoing upper limits (e.g., about 0.1 wt. % to about 3.5 wt. %, about 0.6 wt. % to about 3.1 wt. %, etc.). In some embodiments, the adsorbent is free of or substantially free of alumina. Such embodiments utilizing adsorbents (e.g., silica adsorbents) with low alumina content can advantageously reduce the conversion of methanol to dimethyl ether during regeneration compared to a zeolite-based adsorbent, such as zeolite 4A.

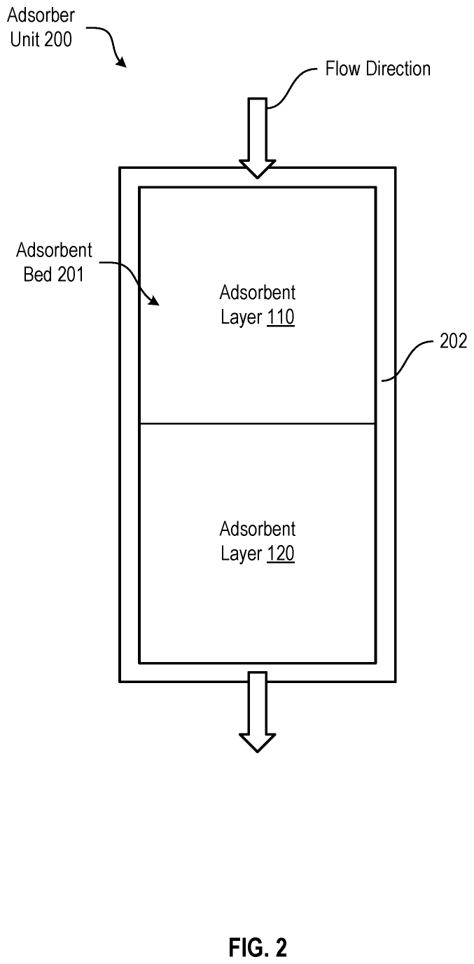

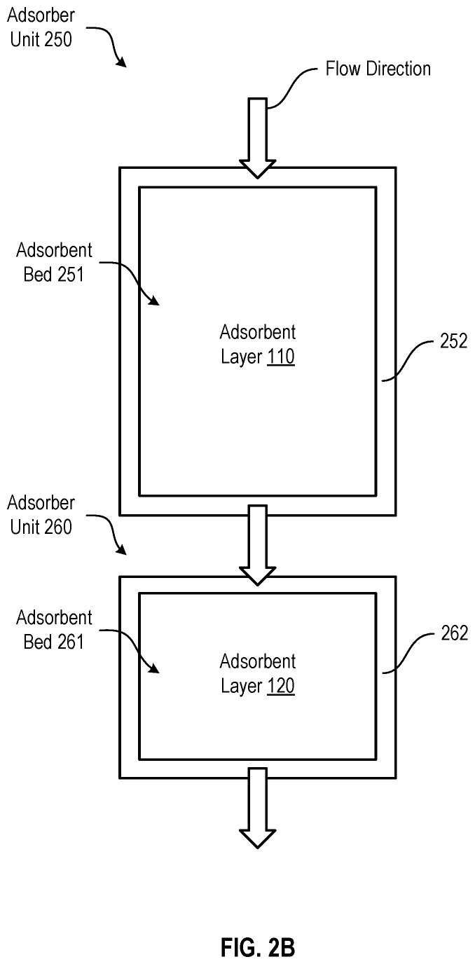

FIG. 2A illustrates an adsorber unit 200 in accordance with at least one embodiment of the disclosure, which represents a variation of the adsorber unit 100. The adsorber unit includes an adsorbent bed 201 includes adsorbent layer 110 and an additional adsorbent layer 120 contained inside a vessel 202. Adsorbent layer 120 is said to be downstream from adsorbent layer 110 based on the depicted flow direction. The relative sizes of the adsorbent layers are not necessarily drawn to scale, though in certain embodiments a weight percent (wt. %) of the adsorbent layer 110 with respect to a total weight of the adsorbent bed 101 (i.e., a total weight of the adsorbent layer 110 and the adsorbent layer 120) may be greater than 50 wt. %, greater than 60 wt. %, greater than 70 wt. %, greater than 80 wt. %, or greater than 90 wt. %.

In some embodiments, the relative sizes of the adsorbent layers 110 and 120 may be adjusted to remove water such that the gas stream (e.g., a natural gas stream) has a water mole fraction that is reduced to less than about 80 ppm, less than about 70 ppm, less than about 60 ppm, less than about 50 ppm, less than about 40 ppm, less than about 30 ppm, less than about 20 ppm, less than about 10 ppm, less than about 5 ppm, less than about 2 ppm by the adsorbent layer 110 prior to the gas stream reaching the adsorbent layer 120, or a water mole fraction of the gas stream leaving the adsorber unit 200 that is below cryogenic specifications (e.g., a water mole fraction below 1 ppm or below 0.1 ppm).

In some embodiments, the relative sizes of the adsorbent layers 110 and 120 may be adjusted to remove methanol such that the gas stream (e.g., a natural gas stream) has a methanol mole fraction that is reduced to less than about 40 ppm, less than about 30 ppm, less than about 20 ppm, less than about 10 ppm, less than about 5 ppm, less than about 2 ppm by the adsorbent layer 110 prior to the gas stream reaching the adsorbent layer 120.

In some embodiments, the adsorbent layer 120 comprises a zeolite. In some embodiments, the adsorbent layer 120 comprises one or more of zeolite A, zeolite X (e.g., zeolite 13X, which is zeolite X that has been exchanged with sodium ions), or zeolite Y. An exemplary adsorbent for use in the adsorbent layer 120 may be Durasorb™ HR4. In some embodiments, the adsorbent layer 120 comprises one or more of zeolite 3A, zeolite 4A or zeolite 5A. In some embodiments, the zeolite is exchanged with any element of columns I and II of the periodic table, such as Li. Na. K. Mg. Ca. Sr. or Ba. Other exemplary adsorbents for the adsorbent layer 120, or a further adsorbent layer downstream from the adsorbent layer 120, include one or more of Durasorb™ BTX, Durasorb™ HC, or Durasorb™ AR.

In some embodiments, the adsorbent layer 120 may comprise a mixture of a zeolite and a microporous adsorbent of silica and/or alumina (e.g., a physical mixture of zeolite particles and microporous adsorbent particles). In some embodiments, the adsorbent layer 120 comprises a gradient of the zeolite and the microporous adsorbent, such that an overall concentration of the microporous adsorbent decreases while the concentration of the zeolite increases along the direction from the layer 110 until an outlet of the vessel 102, or vice versa.

While it is contemplated that a single adsorber unit housing a single adsorbent bed may be used with the various embodiments described herein, two or more adsorbent units may be utilized for the various embodiments described herein. For example. FIG. 2B shows a variant of FIG. 2A, where separate adsorber units 250 and 260 are used, each having separate vessels 252 and 262, respectively, for housing adsorbent beds 251 and 261, respectively. As shown, the adsorbent layer 110 is contained in the vessel 252 of the adsorber unit 250, and the adsorbent layer 120 is contained within the vessel 262 of the adsorber unit 260, with the adsorber unit 260 being downstream from the adsorber unit 250. In some embodiments, the adsorber unit 250 is utilized for heavy hydrocarbon adsorption removal from the gas feed stream, and the adsorber unit 260 is utilized for dehydration of the gas feed stream and/or removal of methanol. Though FIG. 2B provides a simplified view of the adsorber units 250 and 260, it is to be understood that various other components may be present, including heaters, coolers, various valves and connective elements, and controllers to regulate mass flow to, from, and between the adsorber units 250 and 260. Each adsorber unit 250 and 260 may include duplicate vessels and adsorbent beds used to facilitate the implementation of a continuous TSA process.

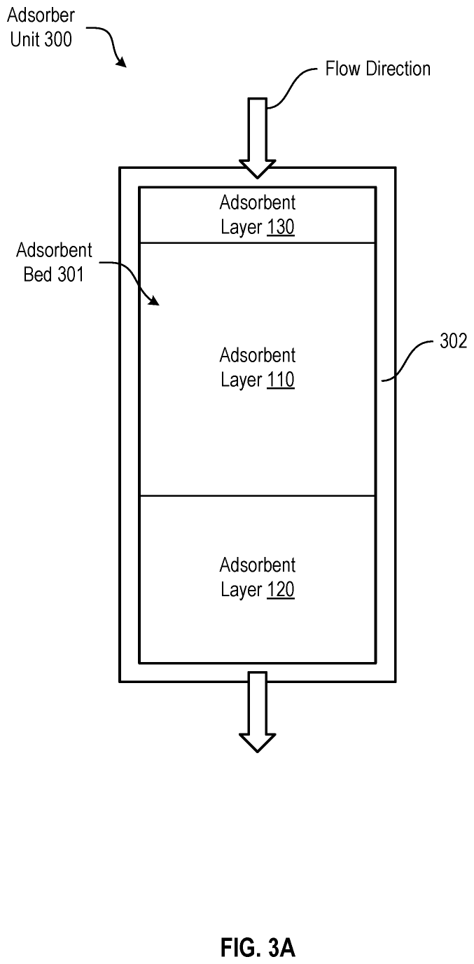

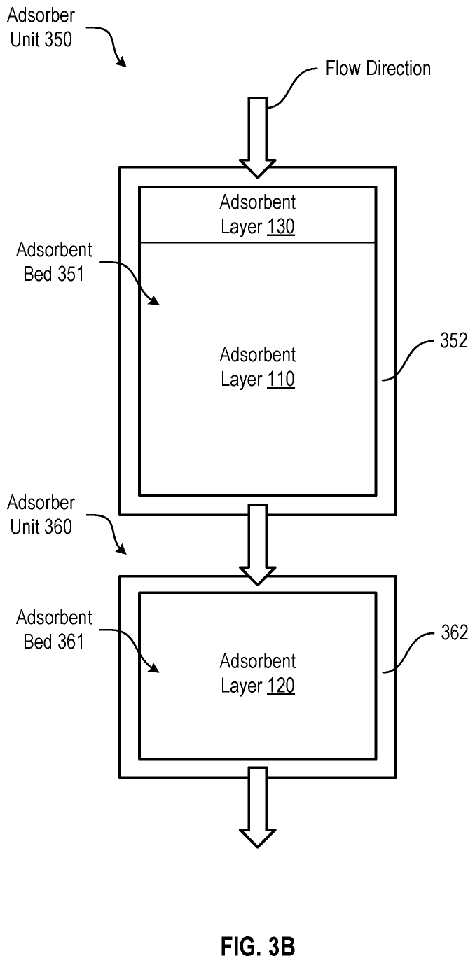

FIG. 3A illustrates a further adsorber unit 300 in accordance with at least one embodiment of the disclosure. The adsorbent bed 301 in the vessel 302 of the adsorber unit 300 is similar to the adsorbent bed 201, except that in addition to the adsorbent layer 110 and adsorbent layer 120, the adsorbent bed 301 further includes an adsorbent layer 130 immediately upstream from the adsorbent layer 110. A further embodiment is also contemplated by modifying the adsorbent bed 101 to include the adsorbent layer 130 immediately upstream from the adsorbent layer 110. In some embodiments, the adsorbent layer 130 comprises a water stable adsorbent, such as Durasorb™ HD (available from BASF), comprising, for example, silica or silica-alumina.

FIG. 3B shows a variant of FIG. 3A, where separate adsorber units 350 and 360 are used, each having separate vessels 352 and 362, respectively, for housing adsorbent beds 351 and 361, respectively. For example, the adsorbent layers 130 and 110 are contained in the vessel 352 of the adsorber unit 350, and the adsorbent layer 120 is contained within the vessel 362 of the adsorber unit 360, with the adsorber unit 360 being downstream from the adsorber unit 350. In some embodiments, each of the adsorbents 110, 120, and 130 may be contained within separate vessels of separate adsorber units. As discussed above with respect to FIG. 1, duplicate adsorbent beds and vessels may be present in each of the adsorber units 350 and 360 to facilitate the implementation of a continuous TSA process.

It is contemplated that a dual- or multi-unit configuration could be applied to any of the adsorber units 100, 200, or 300. In some embodiments, for embodiments for which the adsorbent beds are part of a TSA process, a cycle time may vary for different adsorber units in a multi-unit configuration. For example, with reference to FIG. 2B, the adsorber unit 250 (for which the adsorbent bed 251 may contain, for example, an amorphous silica adsorbent, an amorphous silica-alumina adsorbent, or a high-silica zeolite adsorbent) may be subject to a cycle time of less or equal to about 8 hours, about 7 hours, about 6 hours, about 5 hours, about 4 hours, about 3 hours, about 2 hours, or about 1 hour. The adsorber unit 260 (for which the adsorbent bed 261 may contain, for example, a zeolite) may be subject to a cycle time that is longer than that of the adsorber unit 250, such as greater than 10 hours and up to 24 hours, up to 48 hours, or up to 72 hours. Similar variations in the cycle times may be applied to the configuration of FIG. 3B.

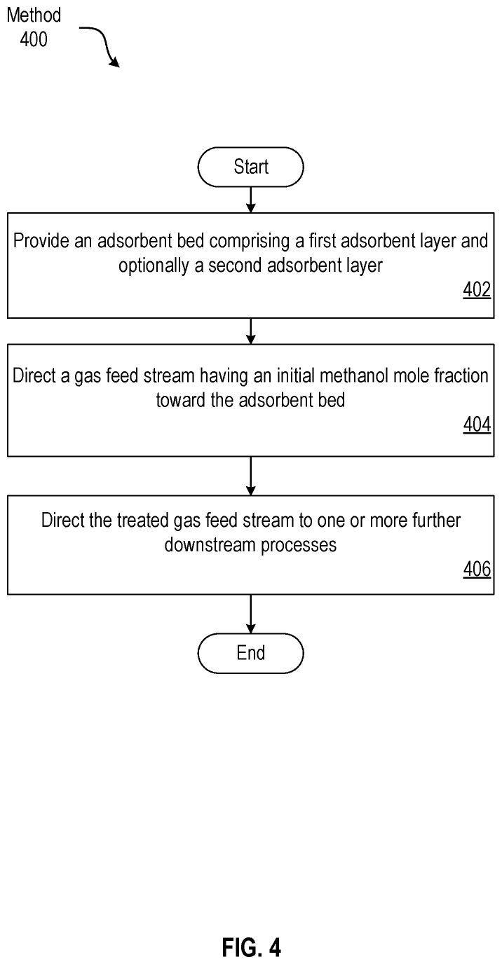

FIG. 4 illustrates a method 400 of treating a gas stream (e.g., a natural gas stream) to remove methanol and reduce or eliminate formation of dimethyl ether, for example, during a regeneration cycle in accordance with an embodiment of the disclosure. At block 402, an adsorbent bed (e.g., any of adsorbent beds 101, 201, 301, or modifications thereof) of an adsorber unit is provided, the adsorbent bed comprising a first adsorbent layer (e.g., the adsorbent layer 110) and optionally a second adsorbent layer (e.g., the adsorbent layer 120). In some embodiments, the adsorbent bed comprises a third adsorbent layer (e.g., the adsorbent layer 130).

In at least on embodiment, the alumina content of the first adsorbent layer is about 3.0 wt. % or less, about 2.9 wt. % or less, about 2.8 wt. % or less, about 2.7 wt. % or less, about 2.6 wt. % or less, about 2.5 wt. % or less, about 2.4 wt. % or less, about 2.3 wt. % or less, about 2.2 wt. % or less, about 2.1 wt. % or less, about 2.0 wt. % or less, about 1.9 wt. % or less, about 1.8 wt. % or less, about 1.7 wt. % or less, about 1.6 wt. % or less, about 1.5 wt. % or less, about 1.4 wt. % or less, about 1.3 wt. % or less, about 1.2 wt. % or less, about 1.1 wt. % or less, about 1.0 wt. % or less, 0.9 wt. % or less, about 0.8 wt. % or less, about 0.7 wt. % or less, about 0.6 wt. % or less, about 0.5 wt. % or less, about 0.4 wt. % or less, about 0.3 wt. % or less, about 0.2 wt. % or less, about 0.1 wt. % or less. In at least one embodiment, the first adsorbent layer is substantially free of alumina.

In at least one embodiment, the first adsorbent layer comprises a microporous adsorbent comprising amorphous silica.

In at least one embodiment, the first adsorbent bed further comprises the second adsorbent layer downstream from the first adsorbent layer. In at least one embodiment, the second adsorbent layer comprises a zeolite. In at least one embodiment, the zeolite comprises one or more of zeolite A, zeolite X, or zeolite Y. In at least one embodiment, the second adsorbent layer comprises one or more of zeolite 3A, zeolite 4A or zeolite 5A. In at least one embodiment, the second adsorbent layer comprises zeolite 4A. In at least one embodiment, the zeolite is exchanged with an element selected from Li, Na, K, Mg, Ca. Sr, or Ba.

In at least one embodiment, the method further comprises directing the gas stream from the first adsorber unit toward an additional adsorbent bed of an additional adsorber unit, the additional adsorbent bed comprising the second adsorbent layer comprising the zeolite. In at least one embodiment, the method is performed as part of a dehydration process.

At block 404, a gas feed stream having an initial methanol mole fraction is directed toward the adsorbent bed of the adsorber unit. In some embodiments, the gas feed stream comprises a natural gas stream. In some embodiments, the gas feed stream comprises predominately methane (at least 50% methane on a molar basis). In some embodiments, the gas feed stream comprises predominately CO₂ (at least 50% CO₂ on a molar basis). In some embodiments, the contact is performed as part of a TSA process. The TSA process may have an adsorption cycle time of less or equal to about 8 hours, about 7 hours, about 6 hours, about 5 hours, about 4 hours, about 3 hours, about 2 hours, or about 1 hour.

The gas feed stream may have an initial methanol mole fraction, and initial water mole fraction, and an initial C5+ or C6+ hydrocarbon mole fraction prior to entering the adsorbent bed and contacting the first adsorbent layer. After passing through the first adsorbent layer, the gas feed stream has a reduced methanol mole fraction and/or a reduced water mole fraction compared to the initial methanol mole fraction and initial water mole fraction, respectively, when the gas feed stream reaches the second adsorbent layer. In some embodiments, block 404 corresponds to an adsorption step in an adsorption cycle in a TSA process. In some embodiments, the reduced methanol mole fraction and/or the reduced water mole fraction are/is maintained for at least 90% of the duration of the adsorption step. That is, the second adsorbent layer, which is less hydrothermally stable than the first adsorbent layer, is contacted with less methanol and/or water than the first adsorbent layer, which increases the overall lifetime of the second adsorbent layer over several TSA cycles. In some embodiments, the reduced water methanol mole fraction and/or the reduced water mole fraction are/is maintained for at least 95%, at least 96%, at least 97%, at least 98%, at least 99%, or 100% of the duration of the adsorption step.

In some embodiments, the initial methanol mole fraction is from about 50 ppm to about 1000 ppm, from about 100 ppm to about 1000 ppm, from about 150 ppm to about 1000 ppm, from about 250 ppm to about 1000 ppm, from about 350 ppm to about 1000 ppm, or from about 450 ppm to about 1000 ppm.

In at least one embodiment, the method further comprises directing, during the regeneration cycle, at least a portion of the treated gas stream through the adsorbent bed of the first adsorber unit, where a conversion of total methanol adsorbed in the adsorbent bed into dimethyl ether for the regeneration cycle is less than 3%, less than 2%, less than 1%, less than 0.5%, or less than 0.2%.

In at least one embodiment, a methanol mole fraction of the gas stream is reduced to about 40 ppm or less, about 30 ppm or less, about 20 ppm or less, about 10 ppm or less, about 5 ppm or less, or about 2 ppm or less prior to the gas stream contacting the second adsorbent layer.

In at least one embodiment, a water mole fraction of the gas stream is reduced to about 80 ppm or less, about 70 ppm or less, about 60 ppm or less, about 50 ppm or less, about 40 ppm or less, about 30 ppm or less, about 20 ppm or less, about 10 ppm or less, about 5 ppm or less, or about 2 ppm or less prior to the gas stream contacting the second adsorbent layer.

In at least one embodiment, a water mole fraction of the gas stream is reduced to about 1 ppm or less prior to the gas stream leaving the second adsorber unit.

In at least one embodiment, a final methanol mole fraction of the gas stream leaving the adsorber unit is about 20 ppm or less, about 15 ppm or less, about 10 ppm or less, about 5 ppm or less, about 4 ppm or less, about 3 ppm or less, about 2 ppm or less, about 1 ppm or less, about 0.5 ppm or less, about 0.4 ppm or less, about 0.3 ppm or less, about 0.2 ppm or less, or below 0.1 or less.

In at least one embodiment, a water mole fraction of the gas stream is about 80 ppm or less, about 70 ppm or less, about 60 ppm or less, about 50 ppm or less, about 40 ppm or less, about 30 ppm or less, about 20 ppm or less, about 10 ppm or less, or about 5 ppm or less.

In some embodiments, the reduced methanol mole fraction is less than about 90%, less than about 80%, less than about 70%, less than about 60%, less than about 50%, less than about 40%, less than about 30%, less than about 20%, less than about 10%, less than about 9%, less than about 8%, less than about 7%, less than about 6%, less than about 5%, less than about 4%, less than about 3%, less than about 2%, or less than about 1% of the initial methanol mole fraction.

In some embodiments, the reduced methanol mole fraction is maintained for 100% of the duration of the adsorption step.

In some embodiments, the reduced water mole fraction is less than or equal to about 90% of the initial water mole fraction. In some embodiments, the reduced water mole fraction is less than about 80%, about 70%, about 60%, about 50%, about 40%, about 30%, about 20%, about 10%, about 9%, about 8%, about 7%, about 6%, about 5%, about 4%, about 3%, about 2%, or about 1% of the initial water mole fraction. In some embodiments, the reduced water mole fraction is less than about 20% of the initial water mole fraction. In some embodiments, the initial water mole fraction is from about 500 ppm to about 1500 ppm, while the reduced water mole fraction is less than or equal to about 500 ppm, about 450 ppm, about 400 ppm, about 350 ppm, about 300 ppm, about 250 ppm, about 200 ppm, about 150 ppm, about 100 ppm, about 50 ppm, about 40 ppm, about 30 ppm, about 20 ppm, about 10 ppm, or about 5 ppm. In other embodiments, the reduced water mole fraction is less than or equal to about 100 ppm, about 50 ppm, about 10 ppm, about 9 ppm, about 8 ppm, about 7 ppm, about 6 ppm, about 5 ppm, about 4 ppm, about 3 ppm, about 2 ppm, or about 1 ppm.

In some embodiments, the gas feed stream has an initial C6+ hydrocarbon mole fraction prior to entering the adsorbent bed that is from about 500 ppm to about 1500 ppm. The gas feed stream may have a reduced C6+ hydrocarbon mole fraction after exiting the adsorbent bed that less than or equal to about 450 ppm, about 400 ppm, about 350 ppm, about 300 ppm, about 250 ppm, about 200 ppm, about 150 ppm, about 100 ppm, about 50 ppm, about 40 ppm, about 30 ppm, about 20 ppm, about 10 ppm, about 5 ppm, about 4, about 3 ppm, about 2 ppm, or about 1 ppm. The gas feed stream may have a reduced C6+ hydrocarbon mole fraction after contacting the first adsorbent layer but prior to contacting the second adsorbent layer that less than or equal to about 450 ppm, about 400 ppm, about 350 ppm, about 300 ppm, about 250 ppm, about 200 ppm, about 150 ppm, about 100 ppm, about 50 ppm, about 40 ppm, about 30 ppm, about 20 ppm, about 10 ppm, about 5 ppm, about 4, about 3 ppm, about 2 ppm, or about 1 ppm.

In some embodiments, one or more components of the hydrocarbons in the gas feed stream is reduced by 100%. 90%, 80%, 70%, 60%, 50%, 40%, 30%. 20%, 10%, or 5% on a molar basis relative to an initial concentration of that component in the gas feed stream, with the one or more components being selected from benzene. C9 hydrocarbons. C8 hydrocarbons. C7 hydrocarbons. C6 hydrocarbons, or C5 hydrocarbons. That is, for a given component in the gas feed stream (e.g., benzene), a concentration of the component in the gas feed stream after passing through the adsorbent bed will be reduced by a specific amount on a molar basis relative to the initial concentration.

At block 406, the treated gas feed stream is directed to one or more further downstream processes, such as additional adsorption steps. In some embodiments, where the gas feed stream is a natural gas stream, a downstream process may be forming a liquefied natural gas product from the gas feed stream if the treated gas feed stream meets cryogenic specifications. For example, final water mole fraction of the gas feed stream after leaving the adsorbent bed may be below 1 ppm or below 0.1 ppm. In some embodiments, the downstream process may be forming a natural gas liquid product from the natural gas stream after leaving the adsorber unit. In at least one embodiment, the method further comprises directing the natural gas stream after leaving the adsorber unit to a natural gas pipeline.

In at least one embodiment, the first adsorbent bed is thermally regenerated during the regeneration cycle. In some embodiments, the adsorbent bed may be regenerated using a clean dry gas stream, such as a product gas from the adsorbent bed (e.g., a treated stream leaving the adsorbent bed) or a stream external to the adsorber unit of which the adsorbent bed is a part. The term “clean dry gas stream” refers to a stream that contains between 0.1 ppm and 30 ppm water, preferably 0.1 ppm to 10 ppm water, between 0.1 and 30 ppm of methanol, preferably between 0.1 ppm and 10 ppm of methanol, and C5+ hydrocarbon species present at less than 50% of the concentration of the gas feed stream of those corresponding species, preferably present at less than 50% of the concentration of the gas feed stream, and most preferably present at less than 50% of the concentration of the gas feed stream. In some embodiments, if the second adsorbent layer is part of a separate adsorber unit than the first adsorbent layer, a clean dry gas stream from the separate adsorber unit may be used to regenerate the second adsorbent layer.

In some embodiments, the adsorbent bed may be retrofitted or refilled by removing and replacing at least a portion of a previously present adsorbent with one or more of the first adsorbent layer or the second adsorbent layer. Retrofitting can include installing internal insulation into the vessel (e.g., the vessel 102), changing adsorption time, changing heating time, changing cooling time, changing regeneration gas flow rate, and changing regeneration gas temperature. In some embodiments, a zeolite material that has been damaged (e.g., hydrothermally damaged) may be replaced with a zeolite adsorbent (e.g., the adsorbent layer 120) that has not been damaged or still has sufficient adsorption capacity.

### ILLUSTRATIVE EXAMPLES

The following examples are set forth to assist in understanding the disclosure and should not, of course, be construed as specifically limiting the embodiments described and claimed herein. Such variations of the disclosed embodiments, including the substitution of all equivalents now known or later developed, which would be within the purview of those skilled in the art, and changes in formulation or minor changes in experimental design, are to be considered to fall within the scope of the embodiments incorporated herein.

In the following examples, “adsorbent A” refers to an amorphous silica gel adsorbent having an alumina content of 3.1 wt. % based on a total weight of the adsorbent, and “adsorbent B” refers to an amorphous silica gel adsorbent having an alumina content of 0.6 wt. % based on a total weight of the adsorbent.

### Example 1

A vessel containing 117 grams of adsorbent A was fed a stream of methane containing 600 ppm methanol at a pressure of 1280 psia and temperature of 25° C. The methane flow was 29 standard liters per minute (slpm) and the bed was fed the feed gas for a period of 11 hours. After the 11 hours, the bed was depressurized to atmospheric pressure and then N₂ was fed to the bed at a flow rate of 17 slpm. The bed was then heated from 25° C. to 270° C. over the course of 2 hours in a linear ramp of temperature, then the bed was held at 270° C. for an additional 2 hours. Subsequently, the bed was cooled to 25° C. Gas chromatogram analysis was performed on the gas leaving the bed and the amounts of methanol and DME were recorded. The conversion of methanol to DME was then calculated as moles DME measured leaving the bed over the regeneration period divided by the sum of moles of DME measured leaving the bed over the regeneration period and moles of methanol leaving the bed over the regeneration period.

### Example 2

The protocol of Example 1 was repeated, except adsorbent A was replaced with adsorbent B.

Results of the two absorbents are compared in Table 1, revealing that lower alumina content reduced DME formation on regeneration.

TABLE 1
Adsorbent A conversion methanol to DME

| | Conversion |
|---|---|
| Adsorbent | (methanol to DME) |
| A | 7.32% |
| B | 0.28% |

### Example 3

An adsorbent bed 1 inch in diameter was filled with 117 grams of adsorbent Durasorb™ HC. The bed was fed with methane containing approximately 650 ppm of methanol at a pressure of 1280 psia and temperature of 28° C. The methane flow was 29 slpm for a period of 11 hours.

### Example 4

The protocol of Example 3 was repeated, except the adsorbent bed was replaced with an adsorbent bed of an amorphous silica-based microporous adsorbent, having a BET surface area of about 778 m²/g, a micropore surface area of about 139 m²/g (corresponding to a an RMA of about 17.9%), a total pore volume for pores between 500 nm and 20000 nm in diameter, between 5 mm³/g and 50 mm³/g, and a pore volume between 0.4 cm³/g and 0.475 cm³/g.

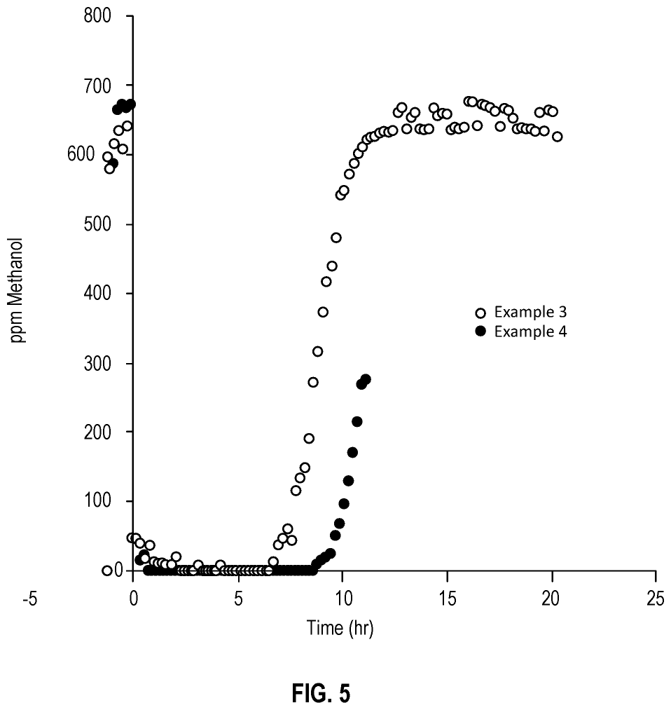

As shown in FIG. 5, the adsorbent bed of Example 4 takes longer to break through to the 200 ppm level than the adsorbent bed of Example 3 (i.e., about 10.5 hours for Example 4 versus 8.5 hours for Example 3).

In the foregoing description, numerous specific details are set forth, such as specific materials, dimensions, processes parameters, etc., to provide a thorough understanding of the embodiments of the present disclosure. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion.

As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A: X includes B: or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form.

Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment, and such references mean “at least one”.

It is to be understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled.

1. A method of treating a gas stream to remove methanol and reduce or eliminate formation of dimethyl ether during a regeneration cycle, the method comprising:
directing, during an adsorption cycle of an adsorption process, the gas stream having an initial methanol mole fraction toward a first adsorbent bed of a first adsorber unit, the first adsorbent bed comprising a first adsorbent layer comprising a silica adsorbent, wherein:
an alumina content of the first adsorbent layer is about 3.1 wt. % or less based on a total weight of the first adsorbent layer, and/or
the initial methanol mole fraction is from about 50 ppm to about 1000 ppm.

2. (canceled)

3. (canceled)

4. The method of claim 1, further comprising:
directing, during the regeneration cycle, at least a portion of the treated gas stream through the first adsorbent bed of the first adsorber unit, wherein a conversion of total methanol adsorbed in the first adsorbent bed into dimethyl ether for the regeneration cycle is less than 7%, wherein the first adsorbent bed is thermally regenerated during the regeneration cycle.

5. (canceled)

6. The method of claim 1, wherein the first adsorbent bed further comprises a second adsorbent layer comprising a zeolite, wherein the second adsorbent layer is downstream from the first adsorbent layer.

7. The method of claim 1, further comprising:
directing the gas stream from the first adsorber unit toward a second adsorbent bed of a second adsorber unit, the second adsorbent bed comprising a second adsorbent layer comprising a zeolite.

8. The method of claim 6, wherein a methanol mole fraction of the gas stream is reduced to about 40 ppm or less, about 30 ppm or less, about 20 ppm or less, about 10 ppm or less, about 5 ppm or less, or about 2 ppm or less prior to the gas stream contacting the second adsorbent layer.

9-13. (canceled)

14. The method of claim 6, wherein the second adsorbent layer comprises zeolite 4A, and wherein the zeolite is exchanged with an element selected from Li, Na, K, Mg, Ca, Sr, or Ba.

15. The method of claim 1, wherein a final methanol mole fraction of the gas stream leaving the first adsorber unit is about 20 ppm or less.

16. (canceled)

17. The method of claim 1, wherein the gas stream is a natural gas stream, and wherein the method further comprises:
forming a liquefied natural gas product from the treated natural gas stream after leaving the first adsorber unit.

18. The method of claim 1, wherein the gas stream is a natural gas stream, and wherein the method further comprises:
forming a natural gas liquid product from the treated natural gas stream after leaving the first adsorber unit.

19. The method of claim 1, wherein the gas stream is a natural gas stream, and wherein the method further comprises:
directing the natural gas stream after leaving the first adsorber unit to a natural gas pipeline.

20. (canceled)

21. (canceled)

22. The method of claim 1, wherein the gas stream comprises predominately CO₂.

23. A method of treating a gas stream to remove methanol and reduce or eliminate formation of dimethyl ether during a regeneration cycle, the method comprising:
directing, during an adsorption cycle of an adsorption process, the gas stream having an initial methanol mole fraction toward a first adsorbent bed of a first adsorber unit, the first adsorbent bed comprising a first adsorbent layer comprising a silica adsorbent, wherein:
the initial methanol mole fraction is from about 250 ppm to about 1000 ppm, and
a conversion of total methanol adsorbed in the first adsorbent bed into dimethyl ether for the regeneration cycle is less than 7%.

24. The method of claim 23, wherein the first adsorbent bed is thermally regenerated during the regeneration cycle.

25. The method of claim 23, wherein the first adsorbent bed further comprises a second adsorbent layer comprising a zeolite, wherein the second adsorbent layer is downstream from the first adsorbent layer.

26. The method of claim 23, further comprising:
directing the gas stream from the first adsorber unit toward a second adsorbent bed of a second adsorber unit, the second adsorbent bed comprising a second adsorbent layer comprising a zeolite.

27. The method of claim 25, wherein a methanol mole fraction of the gas stream is reduced to about 40 ppm or less prior to the gas stream contacting the second adsorbent layer.

28. The method of claim 25, wherein a water mole fraction of the gas stream is reduced to about 80 ppm or less prior to the gas stream contacting the second adsorbent layer.

29. The method of claim 26, wherein a water mole fraction of the gas stream is reduced to about 1 ppm or less prior to the gas stream leaving the second adsorber unit.

30-41. (canceled)

42. A thermal swing adsorption system configured to perform the method of claim 1.

43. A natural gas purification system comprising the thermal swing adsorption system of claim 42.
